(19) United States
(12) Patent Application Publication
(10) Pub. No.: US 2011/0294996 A1
(43) Pub. Date: Dec. 1, 2011

(54) QUANTUM UNIT OF INHERITANCE

(76) Inventors: LANE BERNARD SCHEIBER, Annandale, VA (US); LANE BERNARD SCHEIBER, II, Grosse Ile, MI (US)

(21) Appl. No.: 12/790,932

(22) Filed: May 31, 2010

(51) Int. Cl. C07H 21/00

(52) U.S. Cl. 536/23.1

(57) ABSTRACT

Quantum genes have a unique identifier assigned to them. By identifying genetic material with a unique identifier a means of locating specific genetic material is plausible. Delivering such quantum genes, that contain a unique identifier, to specific cell types provides a means of inserting specific genetic information into the cell's nuclear deoxyribonucleic acid that can be readily located by the cell's nuclear transcription complexes. These medically therapeutic quantum genes are intended to provide a wide variety of medical therapeutic options to clinicians.

### CROSS-REFERENCE TO RELATED APPLICATION

None.

### STATEMENT REGARDING SPONSORED RESEARCH OR DEVELOPMENT

None.

### REFERENCE TO SEQUENCE LISTING, A TABLE, OR COMPUTER LISTING COMPACT DISC APPENDIX

Not applicable.

©2010 Lane B. Scheiber and Lane B. Scheiber II. A portion of the disclosure of this patent document contains material which is subject to copyright protection. The copyright owners have no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

1. Field of the Invention

This invention relates to any medical device associated with gene therapy where the gene therapy is conducted with genetic information labeled with a unique identifying code.

2. Description of Background Art

The central dogma of microbiology dictates that in the nucleus of a biologically active cell, genes are transcribed to produce messenger ribonucleic acid molecules (mRNAs), these mRNAs migrate to the cytoplasm where they are translated to produce proteins. One of the great unknowns that has challenged the study of microbiology is the subject of understanding of how the genes, comprising the genome of a species, are organized such that the nuclear transcription machinery can efficiently locate specific transcribable genetic information and instructions that the cell requires to maintain itself, grow and conduct cell replication. Decoding the means as to how the genetic information contained in the nuclear deoxyribonucleic acid (DNA) of a cell is organized, helps to further the efforts to produce an effective gene therapy treatment strategy. Understanding the basis of genetic instruction code information stored in a cell's DNA and utilizing such knowledge of labeling and cataloging of genetic information, makes inserting biologic instruction into the DNA of cells a practical and effective means of treating a wide scope of medical conditions.

The human genome is comprised of deoxyribonucleic acid (DNA) separated into 46 chromosomes. The chromosomes are further subdivided into genes. Genes represent units of transcribable DNA. Transcription of the DNA refers to generating one or more of a variety of RNA molecules. Regarding the human genome, currently it is estimated that 5% of the total nuclear DNA is thought to represent genes and 95% is thought to represent redundant non-gene genetic material. The DNA genome in a cell is therefore comprised of transcribable genetic information and nontranscribable genetic information. Transcribable genetic information represent the segments of DNA that when transcribed by transcription machinery yield RNA molecules, usually in a precursor form that require modification before the RNA molecules are capable of being translated. The nontranscribable genetic information represent segments that act as either points of attachment for the transcription machinery or act as commands to direct the transcription machinery or act as spacers between transcribable segments of genetic information or have no known function at this time. A segment of nontranslatable DNA that is coded as a STOP command, under the proper circumstances, will cause the transcription machinery to cease transcribing the DNA at that point. A segment of DNA coded to signal a REPEAT command, will cause the transcription machinery to repeat its transcription of a segment of genetic information. The term ‘genetic information’ refers to a sequence of nucleotides that comprise transcribable portions of DNA and nontranscribable portions of DNA. In the DNA, four different nucleotides comprise the nucleotide sequences. The four different nucleotides that comprise the DNA include adenine, cytosine, guanine, and thymine.

Computer programs, commonly utilized in desk top computers, laptop computers, mainframe computers are comprised of a series of software instructions and data. In order for a computer program to run its digital programming in an orderly fashion, each software instruction and each element of data is assigned or associated with a unique identifier such that the software instructions can be carried out in an orderly fashion and each element of data can be efficiently located when there is a need to process the data elements. Similarly, each unit of genetic information, often referred to as a gene, comprising the nuclear DNA of a species genome, must have a unique identifier assigned to it such that the genetic information can be readily located by the transcription machinery and utilized when needed by a cell.

When a gene is to be transcribed, approximately forty proteins assemble together into what is referred to as a transcription complex, which acts as the transcription machinery. The transcription complex forms along a segment of DNA, upstream from the start of the transcribable genetic information. The transcription complex transcribes the genetic information to produce RNA. It is vital to the cell that the transcription complex is able to locate a specific gene amongst the 3 billion base pairs comprising the human genome in an orderly and efficient fashion to enable it to perform functions the cell requires to operate, survive, grow and replicate.

For purposes of this text there are several general definitions. A ‘ribose’ is a five carbon or pentose sugar (C₅H₁₀O₅) present in the structural components of ribonucleic acid, riboflavin, and other nucleotides and nucleosides. A ‘deoxyribose’ is a deoxypentose (C₅H₁₀O₄) found in deoxyribonucleic acid. A ‘nucleoside’ is a compound of a sugar usually ribose or deoxyribose with a nitrogenous base by way of an N-glycosyl link. A ‘nucleotide’ is a single unit of a nucleic acid, composed of a five carbon sugar (either a ribose or a deoxyribose), a nitrogenous base and a phosphate group. There are two families of ‘nitrogenous bases’, which include: pyrimidine and purine. A ‘pyrimidine’ is a six member ring made up of carbon and nitrogen atoms; the members of the pyrimidine family include: cytosine (C), thymine (T) and uracil (U). A ‘purine’ is a five-member ring fused to a pyrimidine type ring; the members of the purine family include: adenine (A) and guanine (G). A ‘nucleic acid’ is a polynucleotide which is a biologic molecule such as ribonucleic acid or deoxyribonucleic acid that enable organisms to reproduce. A ‘ribonucleic acid’ (RNA) is a linear polymer of nucleotides formed by repeated riboses linked by phosphodiester bonds between the 3-hydroxyl group of one and the 5-hydroxyl group of the next; RNAs are single stranded macromolecules comprised of a sequence of nucleotides, these nucleotides are generally referred to by their nitrogenous bases, which include: adenine, cytosine, guanine and uracil. The term macromolecule refers to any very large molecule. RNAs are subset into different types which include messenger RNA (mRNA), transport RNA (tRNA), ribosomal RNA (rRNA) and a variety of small RNAs. Messenger RNAs act as templates to produce proteins. A ribosome is a complex comprised of rRNAs and proteins and is responsible for the correct positioning of mRNA and charged tRNA to facilitate the proper alignment and bonding of amino acids into a strand to produce a protein. A ‘charged’ tRNA is a tRNA that is carrying an amino acid. Ribosomal RNA (rRNA) represents a subset of RNAs that form part of the physical structure of a ribosome. Small RNAs include snoRNA, U snRNA, and miRNA. The snoRNAs modify precursor rRNA molecules. U snRNAs modify precursor mRNA molecules. The miRNA molecules modify the function of mRNA molecules.

A ‘deoxyribose’ is a deoxypentose (C₅H₁₀O₄) sugar. Deoxyribonucleic acid (DNA) is comprised of three basic elements: a deoxyribose sugar, a phosphate group and nitrogen containing bases. DNA is a macromolecule made up of two chains of repeating deoxyribose sugars linked by phosphodiester bonds between the 3-hydroxyl group of one and the 5-hydroxyl group of the next; the two chains are held antiparallel to each other by weak hydrogen bonds. DNA strands contain a sequence of nucleotides, which include: adenine, cytosine, guanine and thymine. Adenine is always paired with thymine of the opposite strand, and guanine is always paired with cytosine of the opposite strand; one side or strand of a DNA macromolecule is the mirror image of the opposite strand. Nuclear DNA is regarded as the medium for storing the master plan of hereditary information.

Genes are considered segments of the DNA that represent units of inheritance.

A chromosome exists in the nucleus of a cell and consists of a DNA double helix bearing a linear sequence of genes, coiled and recoiled around aggregated proteins, termed histones. The number of chromosomes varies from species to species. Most human cells carries twenty two pairs of chromosomes plus two sex chromosomes; two ‘x’ chromosomes in women and one ‘x’ and one ‘y’ chromosome in men. Chromosomes carry genetic information in the form of units which are referred to as genes. The entire nuclear genome, forty six chromosomes, is comprised of 3 billion base pairs (bp) of nucleotides.

Mitochondria possess numerous circular DNA. The limited information stored in mitochondrial DNA is thought to assist the mitochondria in producing the enzymes needed to convert glucose to adenosine triphosphate.

Various standard definitions of a gene exist. Per Stedman's Medical Dictionary, 24^{th} edition, copyright 1982: The functional unit of heredity. Each gene occupies a specific place or locus on a chromosome, is capable of reproducing itself exactly at cell division, and is capable of directing the formation of an enzyme or other protein. The gene as a functional unit probably consists of a discrete segment of purine (adenine and guanine) and pyrimidine (cytosine and thymine) bases in the correct sequence to code the sequence of amino acids needed to form a specific peptide. Protein synthesis is mediated by molecules of messenger RNA formed on the chromosome with the gene unit of DNA acting as a template, which then pass into the cytoplasm and become oriented on the ribosomes where they in turn act as templates to organize a chain of amino acids to form a peptide. Genes normally occur in pairs in all cells except gametes as a consequence of the fact that all chromosomes are paired except the sex chromosomes (x and y) of the male.’

Per Dorland's Pocket Medical Dictionary, 23^{rd} edition, copyright 1982 the definition of ‘gene’ is ‘the biologic unit of heredity, self-producing, and located at a definite position (locus) on a particular chromosome.’

Per the text Understanding Biology, Second Edition, Peter Raven, George Johnson, Mosby, copyright 1991: ‘Gene: The basic unit of heredity. A sequence of DNA nucleotides on a chromosome that encodes a polypeptide or RNA molecule and so determines the nature of an individual's inherited traits.’

Per The New Oxford American Dictionary, Second Edition, copyright 2005: ‘Gene: A unit of heredity that is transferred from a parent to offspring and is held to determine some characteristic of the offspring: proteins coded directly by genes. In technical use: a distinct sequence of nucleotides forming part of a chromosome, the order of which determines the order of monomers in a polypeptide or nucleic acid molecule which a cell (or virus) may synthesize.’

Per MedicineNet.com. (Current as of the time of this publication): According to the official Guidelines for Human Gene Nomenclature, a ‘gene’ is defined as “a DNA segment that contributes to phenotype/function. In the absence of demonstrated function a gene may be characterized by sequence, transcription or homology.” DNA: Genes are composed of DNA, a molecule in the memorable shape of a double helix, a spiral ladder. Each rung of the spiral ladder consists of two paired chemicals called bases. There are four types of bases. They are adenine (A), thymine (T), cytosine (C), and guanine (G). As indicated, each base is symbolized by the first letter of its name: A, T, C, and G. Certain bases always pair together (AT and GC). Different sequences of base pairs form coded messages. The gene: A gene is a sequence (a string) of bases. It is made up of combinations of A, T, C, and G. These unique combinations determine the gene's function, much as letters join together to form words. Each person has thousands of genes—billions of base pairs of DNA or bits of information repeated in the nuclei of human cells—which determine individual characteristics (genetic traits).’

Per Wikipedia.com, referenced to: Group of the Sequence Ontology consortium, coordinated by K. Eilbeck, cited in H. Pearson. (2006). Genetics: what is a gene? Nature, 441, 398-401 (Current as of the time of this publication): A modern working definition of a gene is ‘a locatable region of genomic sequence, corresponding to a unit of inheritance, which is associated with regulatory regions, transcribed regions, and or other functional sequence regions.’

The above definitions of a ‘gene’ are fairly detailed and at present time generally universally accepted in the science and medical communities as representing the definition of a gene. There is a distinct lack of any previous reference in the medical science literature to a unique identifier associated with genetic material.

Current gene theory is derived from Gregor Mendel (1822-1884), who discovered the basic principles of heredity by breeding garden peas at the abbey where he resided, while teaching at Brunn Modern School. Gregor Mendel built and documented a model of inheritance, often referred to as Mendelian genetics, that has acted as the foundation of modern genetics. Gregor Mendel documented changes in characteristics of the plants he grew and described the physical traits as being related to ‘heritable factors’. Over time Mendel's term ‘heritable factor’ has been replaced by the terms ‘gene’ and ‘allele’. Much of what the current term of a ‘gene’ describes remains related to and distinctly linked to the physical traits of the live organisms they describe.

Per J. K. Pal, S. S. Ghaskabi, Fundamentals of Molecular Biology, 2009: ‘The central dogma of molecular biology . . . states that the genes present in the genome (DNA) are transcribed into mRNAs, which are then translated into polypeptides or proteins, which are phenotypes.’ ‘Genome, thus, contains the complete set of hereditary information for any organism and is functionally divided into small parts referred to as genes. Each gene is a sequence of nucleotides representing a single protein or RNA. Genome of a living organism may contain as few as 500 genes as in case of Mycoplasma, or as many as 30,000 genes as in case of human beings.’

Current computer technology utilizes the binary numeric language. Every task a computer performs is related to the language of ‘ones’ and ‘zeros’. Transistors that comprise the inside of computer chips are either turned ‘on’ representing a ‘one’ or turned ‘off’ representing a ‘zero’. At the core of all computer programs is the machine language of ‘ones’ and ‘zeros’. The most sophisticated central processing unit (CPU) in the world only reads and processes the language of ‘ones’ and ‘zeros’. All text, all pictures, all video, all sound and music is diluted down to the form of ‘ones’ and ‘zeros’, and consequently all of the computing and storage power of a computer is performed by the computer language of ‘ones’ and ‘zeros’.

The nucleus of a biologically active cell arguably possesses the most sophisticated and well organized processing power in the world. To run such a powerful processing unit, a form of biologic computer language would seem to be a necessary foundation by which to transfer stored information from the DNA to the remainder of the biologically active portions of a cell as needed. Given that the DNA comprising the chromosomes and mitochondrial DNA are both comprised of four different nucleotides including adenosine, cytosine, guanine and thymine, and RNA is comprised of four nucleotides including adenosine, cytosine, guanine and uracil (uracil in place of thymine), it appears evident the biologic computer language used by a cell's genome is an information language derived from base-four mathematics. Instead of current computer technology utilizing binary computer code comprised of ‘ones’ and ‘zeros’, the DNA and RNA in a biologically active cell utilize an information language comprised of ‘zeros’, ‘one's’, two's’ and ‘threes’ to store and transfer information, which in effect represents a base-four language or quaternary language.

The above definitions of a ‘gene’ refer to genes residing in a specific place or locus on a chromosome. Identifying that a gene is present in a particular location is obvious to the human observer, but from a functional standpoint for cell biology this does not necessarily help a cell find or use the information stored in the nucleotide sequence of a particular gene. To rely on location alone, as a means of identifying a gene, would put the function of the entire genome at peril of failure if even a single base pair of nucleotides were added or deleted from the genome. To this point, no discussion regarding genes being organized utilizing a coding system of any form within the genome, other than the mention of physical location in a chromosome, has been made in the medical literature.

The current understanding of the actual biologic structure of a gene is far more elaborate than the standard definition of a gene leads a casual reader to believe; this knowledge has evolved greatly since Gregor Mendel's work in the 19^{th} century. A gene appears to be comprised of a number of segments loosely strung together along a particular section of DNA. In general there are at least three global segments associated with a gene which include: (1) the Upstream 5′ flanking region, (2) the transcriptional unit and (3) the Downstream 3′ flanking region.

The Upstream 5′ flanking region is comprised of the ‘enhancer region’, the ‘promoter-proximal region’, and ‘promoter region’.

The ‘transcriptional unit’ begins at a location designated ‘transcription start site’ (TSS), which is located in a site called the ‘initiator region’ (inR), which may be described in a general form as Py₂CAPy₅. The transcription unit is comprised of the combination of segments of DNA nucleotides to be transcribed into RNA and spacing units known as ‘introns’ that are not transcribed or if transcribed are later removed post transcription, such that they do not appear in the final RNA molecule. In the case of a gene coding for a mRNA molecule, the transcription unit will contain all three elements of the mRNA, which includes: (1) the 5′ noncoding region, (2) the translational region and (3) the 3′ noncoding region. Interspersed between these regions are exons, which will not be transcribed and introns that if transcribed, are removed from the precursor form of mRNA prior to the mRNA reaching its final form. Exons and introns appear to be likened to spacers. The exact role exons and introns play in the transcription process is undetermined.

The Downstream 3′ flanking region contains DNA nucleotides that are not transcribed and may contain what has been termed an ‘enhancer region’. An enhancer region in the Downstream 3′ flanking region may promote the gene previously transcribed to be transcribed again.

On either side of the DNA sequencing comprising a gene and its flanking regions, may be inactive DNA which act as boundaries which have been termed ‘insulator elements’. The term ‘upstream’ refers to DNA sequencing that occurs prior to the TSS if viewed from the 5′ end to the 3′ end of the DNA; where the term ‘downstream’ refers to DNA sequencing located after the TSS.

The ‘enhancer region’ may or may not be present in the Upstream 5′ flanking region. If present in the Upstream 5′ flanking region, the enhancer region helps facilitate the reading of the gene by encouraging formation of the transcription mechanism. An enhancer may be 50 to 1500 base pairs in length occupying a position upstream from the transcription starting site.

The ‘transcription mechanism’, also referred to as ‘the transcription machinery’ or the ‘transcription complex’ (TC), in humans, is reported to be comprised of over forty separate proteins that assemble together to ultimately function in a concerted effort to transcribe the nucleotide sequence of the DNA into RNA. The transcription mechanism includes elements such as ‘general transcription factor Sp1’, ‘general transcription factor NF1’, ‘general transcription factor TATA-binding protein’, ‘TF_{II}D’, ‘basal transcription complex’, and a ‘RNA polymerase protein’ to name only a few of the forty elements that exist. The elements of the transcription mechanism function as (1) a means to recognize the location of the start of a gene, (2) as proteins to bind the transcription mechanism to the DNA such that transcription may occur or (3) as means of transcribing the DNA nucleotide coding to produce a RNA molecule or a precursor RNA molecule.

There are at least three RNA polymerase proteins which include: RNA polymerase I, RNA polymerase II, and RNA polymerase III. RNA polymerase I tends to be dedicated to transcribing genetic information that will result in the formation of rRNA molecules. RNA polymerase II tends to be dedicated to transcribing genetic information that will result in the formation of mRNA molecules. RNA polymerase III appears to be dedicated to transcribing genetic information that results in the formation of tRNAs, small cellular RNAs and viral RNAs.

The ‘promoter proximal region’ is located upstream from the TSS and upstream from the core promoter region. The ‘promoter proximal region’ includes two sub-regions termed the GC box and the CAAT box. The ‘GC box’ appears to be a segment rich in guanine-cytosine nucleotide sequences. The GC box binds to the ‘general transcription factor Sp1’ of the transcription mechanism. The ‘CAAT box’ is a segment which contains the nucleotide sequence ‘GGCCAATCT’ located approximately 75 base pairs (bps) upstream from the transcription start site (TSS). The CAAT box binds to the ‘general transcription factor NF1’ of the transcription mechanism.

The ‘core promoter’ region is considered the shortest sequence within which RNA polymerase II can initiate transcription of a gene The core promoter may include the inR and either a TATA box or a ‘downstream promoter element’ (DPE). The inR is the region designated Py₂CAPy₅ that surrounds the transcription start site (TSS). The TATA box is located 25 base pairs (bps) upstream from the TSS. The TATA box acts as a site of attachment of the TF_{II}D, which is a promoter for binding of the RNA polymerase II molecule. The DPE may appear 28 bps to 32 bps downstream from the TSS. The DPE acts as an alternative site of attachment for the TF_{II}D when the TATA box is not present.

The transcription mechanism or transcription complex appears to be comprised of different elements depending upon whether rRNA is being transcribed versus mRNA or tRNA or small cellular RNA or viral RNA. The proteins that assemble to assist RNA Polymerase I with transcribing the DNA to produce rRNA appear different from the proteins that assemble to assist RNA polymerase II with transcribing the DNA to produce mRNA and from the proteins that assemble to assist RNA polymerase III with transcribing the DNA to produce tRNA, small cellular RNA or viral RNA. A common protein that appears to be present at the initial binding of all three types of RNA polymerase molecules is TATA-binding protein (TBP). TBP appears to be required to attach to the DNA, which then facilitates RNA polymerase to bind to the promoter along the DNA. TBP assembles with TBP-associated factors (TAFs). Together TBP and 11 TAFs comprise the complex referred to as TF_{II}D, which has been previously mentioned in the above text.

Upstream from the TATA box is the ‘initiator element’, which may be considered as part of the ‘core promoter’ region. The initiator element is a segment of the nuclear DNA that binds the basal transcription complex. The basal transcription complex is comprised of a number of proteins that make initial contact with the DNA prior to the RNA polymerase binding to the transcription mechanism. The basal transcription complex is associated with an activator.

An activator is a protein comprised of three components. The three components of the activator include: (1) DNA binding domain, (2) Connecting domain, and (3) Activating domain. When the activator's DNA binding domain attaches to the DNA at a specific point along the DNA, the activator's activating domain then causes the other elements of the transcription mechanism to assemble at this location. Generally the assembly of the other proteins occurs downstream from where the activator's DNA binding domain attached to the DNA. There is evidence that the activator is associated with the activity of small RNAs.

The design of the cell is so complex, all of its functions so diverse and intricate that some form of practical order is necessitated. The genes must be ordered in some fashion, especially in a human, where there are at least 30,000 different genes used by the cells. Some estimates put the total number of genes present in the human nuclear DNA genome to be closer to 100,000. If no means of order existed as to how the genes could be identified, then ‘random circumstance’ would dictate a cell locating a particular portion of genetic information that it requires, at any given time. Randomness tends to favor the occurrence of random events rather than a purposeful order. A ‘random circumstance’ approach to any living cell would tend to favor failure of the cell rather than survival of the cell.

To allow a cell to utilize the biologic information stored in a gene a ‘unique identifier’ needs to be associated with or attached to the gene's specific nucleotide sequence. In the human genome, the cell's transcription mechanism require an organized means to locate and transcribe any given gene's nucleotide sequence amongst the 3 billion nucleotides that reside in the 46 chromosomes that comprise human DNA. Given how the transcription mechanism assembles upstream from the portion of the gene to be transcribed, the nucleotide sequence acting as a unique identifier associated with a specific gene would be positioned upstream from the transcription start site.

The transcription complex (TC) engages the DNA upstream from the genetic information segment the TC transcribes. The unique identifier may be attached directly to the RNA coding segment of genetic material, or there may exist one or more base pairs physically separating the unique identifier and the RNA coding portion of genetic material. Regarding some genes, there may be numerous base pairs separating the unique identifier from the transcribable region of the gene.

For any form of ‘gene therapy’ to work efficiently, medically therapeutic genetic material inserted into the native DNA of a cell needs to be associated with a unique identifier. Attaching a unique identifier to medically therapeutic genetic material is essential in making it possible for the components of a transcription complex to, in a timely organized fashion, locate the exogenous medically therapeutic genetic material, assemble around this exogenous genetic material, and decode the information contained therein. If no such unique identifier is used, then utilization of such exogenous transcribable genetic information occurs based on the occurrence of random events rather than dictated by therapeutic design.

Naturally occurring unique identifiers in the nuclear genome may occur in numerous forms. Since humans share 47% of their DNA with bananas and 95% of their DNA with monkeys, a portion of the unique identifiers associated with genes in the nuclear DNA may not be specific to a human. Unique identifiers may have a global utility, with a portion of the genome of any organism being shared amongst numerous species. The rational would be that once Nature developed an adequate fundamental design for a particular facet of biologic organisms, this information may be shared amongst numerous species that would benefit from the design. An example might be the basic design of a eukaryote cell; this information would be shared amongst all life that utilized the eukaryote cell design rather than each successive multi-celled species having to repeatedly re-invent the design of a eukaryote cell.

In order for the knowledge base of cellular genetics to progress forward, the definition of a gene must be expanded to include the presence of a ‘unique identifier’ associated with each gene present within the DNA. The basis for the presence of this unique identifier (UI) associated with each active gene is so that the cell can locate the biologic information stored in the DNA nucleotide sequencing of the gene. An active gene refers to those genes present in the genome that are utilized by a particular species to support conception, development and maintenance of a species.

Upon adding a unique identifier to a gene, the current term ‘gene’ is thus expanded to the term ‘quantum gene’. The term ‘quantal’ in biology generally refers to an ‘all or nothing’ state or response. The term ‘quantal’ is a derivative of the word quantum. The term ‘quantum’ means a quantity or amount, and a discrete quantity of energy or a discrete bundle of energy or a discrete quantity of electromagnetic radiation.

A ‘quantum gene’ is comprised of a sequence of nucleotides that represents a ‘unique identifier’ physically linked to a sequence of nucleotides that represent a discrete quantity of genetic information; these sequences of nucleotides being comprised of some combination of the nucleotides being referred to by their nitrogenous base as adenine (A), thymine (T), cytosine (C), and guanine (G). The genetic information associated with the above-mentioned unique identifier may be comprised of a portion of transcribable genetic information and a portion of nontranscribable genetic information which together define a specific gene, otherwise referred to as a discrete quantity of genetic information.

Similar to how a gene is described, with regards to a quantum gene, the term ‘upstream’ refers to DNA sequencing that occurs prior to the transcription start site (TSS) if viewed from the 5′ end to the 3′ end of the DNA; where the term ‘downstream’ refers to DNA sequencing located after the TSS.

Similar to the previously described organization of a standard gene found in nuclear DNA, a quantum gene is structured with at least three global segments which include: (1) the Upstream 5′ flanking region, (2) the transcriptional unit and possibly instructional units and (3) the Downstream 3′ flanking region. The ‘unique identifier’ is located in the Upstream 5′ flanking region. The current standard definition of a gene strictly encompasses the concept that a gene is comprised of a segment of nuclear DNA that when transcribed produces RNA. Therefore, the differences between the current standard definition of a ‘gene’ and the definition of a ‘quantum gene’ is that a quantum gene includes both a unique identifier and a segment of nuclear DNA that when transcribed produces RNA. The segment of nuclear DNA that when transcribed produces RNA is comprised of one or more segments of transcribable genetic information that may be accompanied by one or more segments of nontranscribable genetic information. Nontranscribable segments of genetic information include segments that are removed or ignored during the transcription process or segments that act as commands which includes a START code, STOP code or a REPEAT code. When present, a START code signals initiation of the transcription process. When present, a STOP code signals the discontinuation of the transcription process. When present, a REPEAT code signals that the transcription process should repeat the transcription of the segment of DNA that was just transcribed.

Similar to the standard description of a ‘gene’, a quantum gene's Upstream 5′ flanking region is comprised of the ‘enhancer region’, the ‘promoter-proximal region’, and ‘promoter region’.

Similar to the standard description of a ‘gene’, a quantum gene's ‘transcriptional unit’ begins at a location designated ‘transcription start site’ (TSS), which is located in a site called the ‘initiator region’ (inR), which may be described in a general form as Py₂CAPy₅. The transcription unit is comprised of the combination of segments of DNA nucleotides to be transcribed into RNA and spacing units known as ‘exons’ AND ‘introns’, whereby exons represent segments that are not transcribed and introns represent segments that are transcribed but later removed post transcription, such that they do not appear in the final RNA molecule. In the case of a gene coding for a mRNA molecule, the transcription unit will contain all three elements of the mRNA, which includes: (1) the 5′ noncoding region, (2) the translational region and (3) the 3′ noncoding region. Interspersed between these regions are exons, which will not be transcribed and introns that if transcribed, are removed from the precursor form of mRNA prior to the mRNA reaching its final form. Exons and introns present in nuclear DNA appear to be likened to spacers interspersed in the nuclear DNA. The exact role exons and introns play in the transcription process is undetermined.

Similar to the standard description of a gene, with regards to the quantum gene the Downstream 3′ flanking region contains DNA nucleotides that are not transcribed and may contain what has been termed an ‘enhancer region’. An enhancer region in the Downstream 3′ flanking region may promote the gene previously transcribed to be transcribed again.

On either side of the DNA sequencing comprising a gene and a quantum gene are flanking regions which represent inactive DNA, which act as boundaries which have been termed ‘insulator elements’. Insulator elements are areas that are not transcribed to produce RNA. The function of insulator elements, other than acting as boundary markers between differing genes, is unknown.

In nuclear DNA, quantum genes are comprised of a segment of deoxyribonucleic acid where the portion that represents a unique identifier may be separated from the portion that represents transcribable genetic information by a quantity of base pairs of nucleotides that do not represent a unique identifier and do not represent transcribable genetic information. The purpose of the separation of the portion of the unique identifier from the portion of the genetic information by a quantity of base pairs of nucleotides that do not represent a unique identifier and does not represent genetic information may be to act to facilitate a transcription complex attaching to the quantum gene upstream from the portion of the quantum gene that represents genetic information so that transcription of the biologic information associated with the quantum gene may occur at the designated starting point.

The unique identification or identifier of a quantum gene could be in the form of nucleotide sequence that represents a name assigned to the quantum gene, or a number assigned to a quantum gene or the combination of a name and number assigned to a quantum gene. Irrespective of whether the unique identifier incorporated in a quantum gene is considered a ‘name’, or a ‘number’ or a combination of a name or number, the unique identifier is comprised of a sequence of nucleotides linked to the transcribable genetic information for which it acts as a unique identifier; these sequences of nucleotides being comprised of some combination of the nucleotides being referred to by their nitrogenous base as adenine (A), thymine (T), cytosine (C), and guanine (G). It has been estimated that there are as many as 100,000 separate genes stored in the DNA of the 46 chromosomes comprising the human genome. In a base four language, a string of nine nucleotides is needed to code for 256,144 individual genes. If there were over a million quantum genes, then a string of ten nucleotides could be used since ten nucleotides could represent 1,024,576 unique numbers in a base-four number system.

Utilizing a base four number system a string of twenty-five nucleotides would represent the number 1,125,899,906,842,624, which could account for 200,000 different quantum genes in 5 billion different species. Therefore 200,000 different quantum genes could be dedicated to producing a biped form of life. In the human genome 5% of the 3 billion base pairs are considered to represent genes by the current definition of a gene. If 5% of the human genome represents the 100,000 quantum genes in the nuclear DNA, then on average 1500 nucleotides can be dedicated to each gene. If 25 nucleotides are dedicated to a unique address or unique identifier, then there remain 1475 nucleotides, on average, to be utilized for coding the biologic information associated with each of the 100,000 quantum genes estimated to exist in the human genome.

A unique identifier (UI) incorporated in quantum genes could be comprised of a unique number or a unique name or the unique combination of a number and a name. A name might be represented as a single letter or a series of letters. The current convention utilized in science is to apply the four letter alphabet A, C, G, T to represent the four different bases of the nucleotides comprising the DNA, which include adenosine, cytosine, guanine, and thymine respectfully. With regards to RNA, the four letter alphabet A, C, G, U is utilized to represent the bases of the nucleotides which include adenosine, cytosine, guanine, and uracil. Regarding utilizing a unique identifier for DNA, a name could be comprised of a series of letters derived from the four letters A, C, G, and T. Regarding utilizing a unique identifier for purposes of use within an RNA molecule, a unique identifier could be comprised of a series of letters derived from the four letters A, C, G, and U. The current scientific convention does not recognize a mathematical base-four nomenclature regarding DNA or RNA. The unique identifier could be represented as a number. Names can be translated into numbers and vice versa.

In the nuclear DNA, there are several places in the upstream segment of a quantum gene where a segment of twenty-five or more base pairs could exist that acts as the unique identifying code that uniquely identifies the segment of transcribable genetic information. The transcription start site (TSS) is present upstream from a segment of transcribable genetic information. There exists a segment of 25 bps upstream from the TSS that occupies the space along the DNA between the TSS and the TATA box. There exists the downstream promoter element (DPE) 28 bps to 32 bps downstream from the TSS. The DPE acts as an alternative site of attachment for the TF_{II}D when the TATA box is not present. Within the 28 bps to 32 bps of DNA separating the DPE from the TSS may also be a convenient location for a unique identifying code to reside and be associated with the genetic information located just downstream. The cell exists with numerous variability. There exists variation in the arrangement of the elements upstream from the transcribable genetic information, therefore various sites upstream from the transcribable genetic information may function as the unique identifying code for some quantum genes. The unique identifying code may be represented as subsegments of DNA, where subsegments are physically separated from each other, but in combination, the subsegments act in unison to identify a segment of transcribable genetic information.

By delivering quantum genes containing the genetic information required to produce insulin directly to the cells responsible for the production of insulin, the medical treatment of diabetes mellitus is significantly improved. Diabetes mellitus represents a state of hyperglycemia, a serum blood sugar that is higher than what is considered the normal range for humans. Glucose, a six-carbon molecule, is a form of sugar. Glucose is absorbed by the cells of the body and converted to energy by the processes of glycolysis, the Krebs cycle and phosporylation. Insulin, a protein, facilitates the transfer of glucose from the blood into cells. Normal range for blood glucose in humans is generally defined as a fasting blood plasma glucose level of between 70 to 110 mg/dl. For descriptive purposes, the term ‘plasma’ refers to the fluid portion of blood.

Diabetes mellitus is classified as Type One and Type Two. Type One diabetes mellitus is insulin dependent, which refers to the condition where there is a lack of sufficient insulin circulating in the blood stream and insulin must be provided to the body in order to properly regulate the blood glucose level. When insulin is required to regulate the blood glucose level in the body, this condition is often referred to as insulin dependent diabetes mellitus (IDDM). Type Two diabetes mellitus is noninsulin dependent, often referred to as noninsulin dependent diabetes mellitus (NIDDM), meaning the blood glucose level can be managed without insulin, and instead by means of diet, exercise or intervention with oral medications. Type Two diabetes mellitus is considered a progressive disease, the underlying pathogenic mechanisms including pancreatic Beta cell (also often designated as β-Cell) dysfunction and insulin resistance.

The pancreas serves as an endocrine gland and an exocrine gland. Functioning as an endocrine gland the pancreas produces and secretes hormones including insulin and glucagon. Insulin acts to reduce levels of glucose circulating in the blood. Beta cells secrete insulin into the blood when a higher than normal level of glucose is detected in the serum. For purposes of this description the terms ‘blood’, ‘blood stream’ and ‘serum’ refer to the same substance. Glucagon acts to stimulate an increase in glucose circulating in the blood. Beta cells in the pancreas secrete glucagon when a low level of glucose is detected in the serum.

Glucose enters the body and then the blood stream as a result of the digestion of food. The Beta cells of the Islets of Langerhans continuously sense the level of glucose in the blood and respond to elevated levels of blood glucose by secreting insulin into the blood. Beta cells produce the protein ‘insulin’ in their endoplasmic reticulum and store the insulin in vacuoles until it is needed. When Beta cells detect an increase in the glucose level in the blood, Beta cells release insulin into the blood from the described storage vacuoles.

Insulin is a protein. An insulin protein consists of two chains of amino acids, an alpha chain and a beta chain, linked by two disulfide (S—S) bridges. One chain, the alpha chain consists of 21 amino acids. The second chain the beta chain consists of 30 amino acids.

Insulin interacts with the cells of the body by means of a cell-surface receptor termed the ‘insulin receptor’ located on the exterior of a cell's ‘outer membrane’, otherwise known as the ‘plasma membrane’. Insulin interacts with muscle and liver cells by means of the insulin receptor to rapidly remove excess blood sugar when the glucose level in the blood is higher than the upper limit of the normal physiologic range. Recognized functions of insulin include stimulating cells to take up glucose from the blood and convert it to glycogen to facilitate the cells in the body to utilize glucose to generate biochemically usable energy, and to stimulate fat cells to take up glucose and synthesize fat.

Diabetes Mellitus may be the result of one or more factors. Causes of diabetes mellitus may include: (1) mutation of the insulin gene itself causing miscoding, which results in the production of ineffective insulin molecules; (2) mutations to genes that code for the ‘transcription factors’ needed for transcription of the insulin gene in the deoxyribonucleic acid (DNA) to create messenger ribonucleic acid (mRNA) molecules, which facilitate the manufacture of the insulin molecule; (3) mutations of the gene encoding for the insulin receptor, which produces inactive or an insufficient number of insulin receptors; (4) mutation to the gene encoding for glucokinase, the enzyme that phosphorylates glucose in the first step of glycolysis; (5) mutations to the genes encoding portions of the potassium channels in the plasma membrane of the Beta cells, preventing proper closure of the channel, thus blocking insulin release; (6) mutations to mitochondrial genes that as a result, decreases the energy available to be used facilitate the release of insulin, therefore reducing insulin secretion; (7) failure of glucose transporters to properly permit the facilitated diffusion of glucose from plasma into the cells of the body.

A ‘eukaryote’ refers to a nucleated cell. Eukaryotes comprise nearly all animal and plant cells. A human eukaryote or nucleated cell is comprised of an exterior lipid bilayer plasma membrane, cytoplasm, a nucleus, and organelles. The exterior plasma membrane defines the perimeter of the cell, regulates the flow of nutrients, water and regulating molecules in and out of the cell, and has embedded into its structure receptors that the cell uses to detect properties of the environment surrounding the cell membrane. The cytoplasm acts as a filling medium inside the boundaries of the plasma cell membrane and is comprised mainly of water and nutrients such as amino acids, oxygen, and glucose. The nucleus, organelles, and ribosomes are suspended in the cytoplasm. The nucleus contains the majority of the cell's genetic information in the form of double stranded deoxyribonucleic acid (DNA). Organelles generally carry out specialized functions for the cell and include such structures as the mitochondria, the endoplasmic reticulum, storage vacuoles, lysosomes and Golgi complex (sometimes referred to as a Golgi apparatus). Floating in the cytoplasm, but also located in the endoplasmic reticulum and mitochondria are ribosomes. Ribosomes are complex macromolecule structures comprised of ribosomal ribonucleic acid (rRNA) molecules and ribosomal proteins that combine and couple to a messenger ribonucleic acid (mRNA) molecule. The rRNAs and the ribosomal proteins congregate to form a macromolecule structure that surrounds a mRNA molecule. Ribosomes decode genetic information in a mRNA molecule and manufacture proteins to the specifications of the instruction code physically present in the mRNA molecule. More than one ribosome may be attached to a single mRNA at a time.

Proteins are comprised of a series of amino acids bonded together in a linear strand, sometimes referred to as a chain; a protein may be further modified to be a structure comprised of one or more similar or differing strands of amino acids bonded together. Insulin is a protein structure comprised of two strands of amino acids; one strand comprised of 21 amino acids long and the second strand comprised of 30 amino acids, the two strands attached by two disulfide bridges. There are an estimated 30,000 different proteins the cells of the human body may manufacture. The human body is comprised of a wide variety of cells, many with specialized functions requiring unique combinations of proteins and protein structures such as glycoproteins (a protein combined with a carbohydrate) to accomplish the required task or tasks a specialized cell is designed to perform. Forms of glycoproteins are known to be utilized as cell-surface receptors. Messenger RNAs (mRNA) are created by transcription of DNA, they generally migrate to other locations inside the cell and are utilized by ribosomes as protein manufacturing templates. A ribosome is a protein complex that manufactures proteins by deciphering the instruction code located in a mRNA molecule. When a specific protein is needed, pieces of the ribosome complex, which include rRNA molecules and ribosomal proteins, bind around the strand of a mRNA that carries the specific instruction code that will generate the required protein. The ribosome traverses the mRNA strand and deciphers the genetic information coded into the sequence of nucleotides that comprise the mRNA molecule to produce a protein molecule and this process is referred to as translation.

The insulin molecule is a protein produced by Beta cells located in the pancreas. The ‘insulin messenger RNA’ is created in a Beta cell by a polymerase complex transcribing the insulin gene from nuclear DNA in the nucleus of the cell. The native messenger RNA (mRNA) for insulin then travels to the endoplasmic reticulum where numerous ribosomes, comprised of rRNA and ribosomal proteins, engage these mRNA molecules. Many ribosomes may be attached to a single strand of mRNA simultaneously, each generating an identical copy of the protein as dictated by the information encoded in the mRNA. Insulin is produced by ribosomes translating the information in a mRNA molecule coded for the insulin protein, which produce strands of amino acids that are coded for an immature form of the biologically active insulin molecule referred to as ‘pro-insulin’. Once the pro-insulin molecule is generated it then undergoes modification by several enzymes including prohormone convertase one (PC1), prohormone convertase two (PC2) and carboxypeptidase E, which results in the production of a biologically active insulin molecule. Once the biologically active insulin protein is generated it is stored in a vacuole in the Beta cell to await being released into the blood stream.

Insulin receptors, which appear on the surface of cells, offer binding sites for insulin circulating in the blood. When insulin binds to an insulin receptor, the biologic response inside the cell causes glucose to enter the cell and undergo processing in the cytoplasm. Processed glucose molecules then enter the mitochondria. The mitochondria further process the modified glucose molecules to produce usable energy in the form of adenosine triphosphate molecules (ATP). Thirty-eight ATP molecules may be generated from one molecule of glucose during the process of aerobic respiration. ATP molecules are utilized as an energy source by biologic processes throughout the cell.

The current medical therapeutic approach for the management of diabetes mellitus has produced limited results. Patients with diabetes generally struggle with an inadequate production of insulin, or an ineffective release of biologically active insulin molecules, or a release of an insufficient number of biologically active insulin molecules, or an insufficient production of cell-surface receptors, or a production of ineffective cell-surface receptors, or a production of ineffective insulin molecules that are unable to interact properly with insulin receptors to produce the required biologic effect. Type One diabetes requires administration of exogenous insulin. The traditional approach to Type Two diabetes has generally first been to adjust the diet to limit the caloric intake the individual consumes. Exercise is used as an initial approach to both Type One and Type Two diabetes as a means of up-regulating the utilization of fats and sugar so as to reduce the amount of circulating plasma glucose. When diet and exercise are inadequate in properly managing Type Two diabetes, oral medications are often introduced. The action of sulfonylureas, a commonly prescribed class of oral medication, is to stimulate the Beta cells to produce additional insulin receptors and enhance the insulin receptors' response to insulin. Biguanides, another form of oral treatment, inhibit gluconeogenesis, the production of glucose in the liver, thereby attempting to reduce plasma glucose levels. Thiazolidinediones (TZDs) lower blood sugar levels by activating peroxisome proliferator-activated receptor gamma (PPAR-y), a transcription factor, which when activated regulates the activity of various target genes, particularly ones involved in glucose and lipid metabolism. If diet, exercise and oral medications do not produce a satisfactory control of the level of blood glucose in a diabetic patient, exogenous insulin is injected into the body in an effort to normalize the amount of glucose present in the serum. Insulin, a protein, has not successfully been made available as an oral medication to date due to the fact that proteins in general become degraded when they encounter the acid environment present in the stomach.

Despite strict monitoring of blood glucose and potentially multiple doses of insulin injected throughout the day, many patients with diabetes mellitus still experience devastating adverse effects from elevated blood glucose levels. Microvascular damage and elevated tissue sugar levels contribute to such complications as renal failure, retinopathy involving the eyes, neuropathy, and accelerated heart disease despite aggressive efforts to maintain the blood sugar within the physiologic normal range using exogenous insulin by itself or a combination of exogenous insulin and one or more oral medications. Diabetes remains the number one cause of renal failure in the United States. Especially in diabetic patients that are dependent upon administering exogenous insulin into their body, though dosing of the insulin may be four or more times a day and even though this may produce adequate control of the blood glucose level to prevent the clinical symptoms of hyperglycemia; this does not unerringly supplement the body's natural capacity to monitor the blood sugar level minute to minute, twenty-four hours a day, and deliver an immediate response to a rise in blood glucose by the release of insulin from Beta cells as required. The deleterious effects of diabetes may still evolve despite strict and persistent control of the glucose level in the blood stream.

The current treatment of diabetes may be augmented by the unique approach to utilizing modified viruses as vehicles to transport quantum genes into cells in order to increase the production of biologically active insulin. By utilizing modified viruses to transport quantum genes to facilitate and enhance the production of mRNAs, which would then facilitate the assembly of proteins would offer a new treatment option for patients with diabetes.

Present medical care is attempting to utilize viruses to deliver genetic information into cells. Research in the field of gene therapy has involved certain naturally occurring viruses. Some of the common viral vectors that have been investigated include: Adeno-associated virus, Adenovirus, Alphavirus, Epstein-Barr virus, Gammaretrovirus, Herpes simplex virus, Letivirus, Poliovirus, Rhabdovirus, Vaccinia virus. Naturally occurring virus vectors are limited to the naturally occurring external probes that are affixed to the outer wall of the virus. The external probes fixed to the outside wall of a virus virion dictate which type of cell the virus can engage and infect. Therefore, as an example, the function of the adenovirus, a respiratory virus, is strictly limited to engaging and infecting specific lung cells. Used as a medical treatment device, the adenovirus can only deliver gene therapy to specific lung cells, which severely limits this vector's usefulness as a deliver device. The therapeutic function of all naturally occurring viral vectors is limited to delivering a DNA or RNA payload to the cell type the viral vector naturally targets as its host cell.

Cichutek, K., 2001 (U.S. Pat. No. 6,323,031 B1) teaches preparation and use of novel lentiviral SiVagm-derived vectors for gene transfer into selected cell types, specifically into proliferatively active and resting human cells.

Cichutek teaches that it is indeed plausible to re-configure an existing virus and use it as a transport vehicle, though Cichutek's specification and claims are too limited to describe a method that will work for all cell types, if indeed if it will work for any cell type.

Cichutek describes vectors for ‘gene transfer’; in the claims the language that is used is ‘genetic information’. Cichutek's claim 1 of the cited patent states ‘A propagation-incompetent SIVagm vector comprising a viral core and a viral envelope, wherein the viral core comprises a simian immunodeficiency virus (SIVagm) viral core of the African vervet monkey Chlorocebus.’ Cichutek's does not describe in his claims any further details of the intended payload other than the stating ‘SIVagm viral core’ in claim 1; in claims 5 & 6 Cichutek describes only ‘genetic information’. Transfer of ‘genetic information’ dramatically limits the useful application of Cichutek's patent in the treatment of medical diseases.

The necessity for labeling genetic material with a unique identifier has not yet been recognized by those skilled in the art.

For purposes of this text, the term ‘exogenous’ refers to an item which originates outside the boundaries of a particular cell or cell type and is caused to become a part of a particular cell or cell type. The term ‘endogenous’ refers to an item which originates as a part of a particular cell or cell type and remains a part of that particular cell or cell type.

### BRIEF SUMMARY OF THE INVENTION

Quantum genes are comprised of a unique identifier linked to a segment of genetic information, at least a portion of this genetic information coding for the production of one or more ribonucleic acids. By delivering one or more quantum genes to a specific cell type or an array of cell types for installation into the DNA of the cells, the exogenous easily identifiable genetic instructions made available to the cells can be located and transcribed in an efficient manner and thus utilized in one or more specific cell types in a timely fashion. A wide variety of medical conditions are manageable by utilizing this new and unique approach.

### DETAILED DESCRIPTION

The future of medical treatment will be the widespread utilization of quantum genes delivered directly to targeted cell types in the body in order to manage protein deficient states.

For the purposes of this text a ‘quantum gene’ is comprised of a sequence of nucleotides that represents a ‘unique identifier’ physically linked to a sequence of nucleotides that represent a discrete quantity of genetic information; these sequences of nucleotides being comprised of some combination of the nucleotides being referred to by their nitrogenous base as adenine (A), thymine (T), cytosine (C), and guanine (G). The genetic information associated with the above-mentioned unique identifier may be comprised of a portion of transcribable genetic information and a portion of nontranscribable genetic information which together define a specific gene, otherwise referred to as a discrete quantity of genetic information. The nontranscribable segments of a quantum gene may represent segments that act as instructions such as a START code, STOP code and REPEAT code or may help facilitated the attachment of a transcription complex or be simply ignored during the transcription process. Quantum gene molecules can be comprised of a segment of nucleotides where the portion that represents a unique identifier is separated from the portion that represents genetic information by a quantity of base pairs of nucleotides that do not represent a unique identifier and do not represent genetic information. The purpose of the separation of the portion of the unique identifier from the portion of the genetic information by a quantity of base pairs of nucleotides that do not represent a unique identifier and does not represent genetic information is to facilitate a transcription complex attaching to the quantum gene upstream from the portion of the quantum gene that represents genetic information so that transcription of the biologic information associated with the quantum gene may occur.

The genetic information in a quantum gene codes for some combination of protein coding RNA (pcRNA), non-coding RNAs (ncRNA) and spacers. Spacers represent segments of nucleotides that do not code for a RNA molecule. The genetic information in a quantum gene, when transcribed, produces protein coding RNA and non-coding RNA. Protein coding RNAs, usually referred to as messenger RNAs, undergo the process of translation in the cytoplasm of the cell and produce proteins. Non-coding RNAs are highly abundant and functionally important for the cell's operation. Non-coding RNAs have also been referred to by such terms as non-protein-coding RNAs (npcRNA) or non-messenger RNA (nmRNA) or small non-messenger RNA (snmRNA) or functional RNAs (fRNA). The non-coding RNAs include: transfer RNAs (tRNA), ribosomal RNAs (rRNA), small nuclear RNAs (snRNA), small nucleolar RNAs (snoRNA), signal recognition particle RNA (SRP RNA), antisense RNA (aRNA), micro RNA (miRNA), small interfering RNA (siRNA), and Y RNA, telomerase RNA.

Transfer RNAs (tRNA), are RNAs that carries amino acids and deliver them to a ribosome. Ribosomal RNAs (rRNA), are RNAs that couple with ribosomal proteins and participate in translation of mRNA to produce protein molecules. Small nuclear RNAs (snRNA) are RNAs involved in splicing and other nuclear functions. Small nucleolar RNAs (snoRNA) are RNAs involved in nucleotide modification. Signal recognition particle RNA (SRP RNA) are RNAs involved in membrane integration. Antisense RNA (aRNA) are RNAs involved in transcription attenuation, mRNA degradation, mRNA stabilization, and translation blockage. Micro RNA (miRNA) are RNAs involved in gene regulation and have been implicated in a wide range of cell functions including cell growth, apoptosis, neuronal plasticity, and insulin secretion. Small interfering RNA (siRNA) are RNAs involved in gene regulation, often interfering with the expression of a single gene. Y RNA are RNAs involved in RNA processing and DNA replication. Telomerase RNA are RNAs involved in telomere synthesis.

In addition to a unique identifier, a quantum gene is comprised of the biologic instruction code, which when transcribed produce one or more of the same RNA molecules or different RNA molecules. A quantum gene must be comprised of a unique identifier and the genetic material to code for at least one RNA molecule. The definition of a ‘quantum gene’ differs from all previous definitions of a ‘gene’ due to the requirement that the quantum gene must have a unique identifier that accompanies a segment of genetic information. From a medical treatment perspective, the quantum gene's unique identifier allows the genetic information present in the quantum gene to be located by a cell's transcription machinery, once the quantum gene is inserted into a cell's nuclear DNA.

Ribonucleic acid molecules directly transcribed from the DNA or quantum gene, may be precursor ribonucleic acid molecules that require modification by nuclear enzymes prior to being translatable or may be ribonucleic acid molecules which are directly translatable without further modification.

In the DNA there are a number of nucleotides physically existing along the deoxyribonucleic acid between the unique identifier and the transcribable genetic information; or in other terms a number of nucleotides that are not a part of the identification code and are not transcribable, exist downstream from the unique identifier and upstream from the transcribable genetic information.

It is well recognized that within the transcribable genetic information there exist subsegments of nucleotides that are not transcribable and there are subsegments of nucleotides that are transcribed but are not found in the final version of the RNA molecule. Subsegments of transcribable genetic information that are not transcribed are subsegments such as ‘STOP’ codes, which indicate to the transcription complex a potential point at which to cease transcribing the genetic information. Certain factors may influence whether a transcription complex actually ceases transcription at that point or whether the transcription complex continues transcribing when the transcription complex reaches a ‘STOP’ code. Subsegments of nucleotides that are transcribed and appear in the final active form of a RNA are referred to as exons. Subsegments of nucleotides that are transcribed, but do not appear in the final active form of a RNA are referred to as introns. Precursor RNA molecules include both exons and introns. Introns are removed by modification of the initial RNA segment directly transcribed from the transcribable genetic information.

Utilization of the sigma summation symbol to show summation over a series of indexed variables or expression can be represented as:

Σⱼ₌₁ⁿ [K]ⱼ=[K]₁+[K]₂+ . . . +[K]ₙ

An equation to represent a quantum gene would be:

Quantum gene=[unique identifier]+Σₐ₌₀ⁿ[nontranscribable connector nucleotide]ₐ+Σ_{b=1}ⁿ[nucleotide segment transcribable for RNA]_{b}+Σ_{c=0}ⁿ[nontrasncribable spacer nucleotide]_{c}+Σ_{d=0}ⁿ[nontranscribable nucleotide commands]_{d}

Where ‘unique identifier’ represents a number, a name or the combination of a number and a name that the transcription complex utilizes to locate a specific quantum gene amongst the DNA material present in a biologically active cell.

Where ‘nontranscribable connector nucleotide’ represents one or more nucleotides that physically exists between the ‘unique identifier’ and the segment of ‘transcribable genetic information’.

Where a ‘nontranscribable spacer nucleotide’ represents one or more nucleotides comprising the transcribable genetic information that is not transcribed when the transcription complex transcribes the genetic information of the quantum gene.

Where a ‘nontranscribable nucleotide command’ represents one or more nucleotides comprising the transcribable genetic information that is not transcribed when the transcription complex transcribes the genetic information of the quantum gene, but acts as an instruction to the transcription complex to cause the transcription complex to function in a certain manner; examples include a STOP code that causes the transcription complex to cease transcription and a REPEAT code that causes the transcription complex to repeat its transcription of a segment of genetic material.

Where ‘a’ represents the range of ‘zero to any positive whole number.

Where ‘b’ represents the range of ‘one to any positive whole number’.

Where ‘c’ represents the range of ‘zero to any positive whole number’.

Where ‘d’ represents the range of ‘zero to any positive whole number’.

Where the DNA segment that is transcribable for RNA may transcribe RNAs that may exist in a precursor form; such a precursor form may include elements such as introns that are removed following transcription by modifying proteins.

As an example of this method, to treat diabetes mellitus utilizing configurable microscopic medical payload delivery devices to deliver to Beta cells quantum genes that code for messenger RNA that when translated produce insulin molecules, the following production process is followed in the lab: (1) human stem cells are selected. (2) Into the selected stem cells is placed the production genome constructed, in this case, specifically as a means to treat diabetes mellitus. The RNA production genome contains genetic instructions to cause the host stem cells to manufacture the quantum genes to activate the production of the insulin molecules in Beta cells; the biologic instructions to assemble the components into the final form of the configurable microscopic medical payload delivery devices; and the biologic instructions to activate the budding process. (3) Upon insertion of the RNA production genome dedicated to producing a quantum genes configured to activate the genes to generate messenger RNA that will result in the production of insulin, into the host stem cells, host stem cells respond by simultaneously translating the different segments of the RNA production genome to produce the proteins that comprise the exterior protein wall, the inner protein matrix molecules, and the quantum gene to produce insulin. Upon production the gene molecules are packaged into vacuoles and expressed from the host cell. (4) The quantum gene molecules are collected from the nutrient broth surrounding the host stem cells. (5) The quantum stem cells are separated from the nutrient. (6) The configurable quantum gene molecules are suspended in a hypoallergenic liquid medium. (7) The quantum gene molecules are divided into individual quantities to facilitate storage and delivery to physicians and patients. (8) Modified virus vectors or configurable microscopic medical payload delivery devices containing the quantum gene molecules suspended in a hypoallergenic liquid medium are administered to a diabetic patient per injection in a dose that is tailored to receiving patient's requirement to produce sufficient amount of insulin to control the blood sugar. (9) Upon being injected into the body, the modified viruses or the configurable microscopic medical payload delivery devices migrate to the Beta cells located in the Islets of Langerhans by means of the patient's blood stream. (10) Upon the modified virus or the configurable microscopic medical payload delivery devices reaching the Beta cells, the configurable microscopic medical payload delivery devices engage the cell-surface receptors located on the Beta cells and insert the payload of quantum genes they carry into the Beta cells. The payload of quantum genes migrate to the nucleus of the Beta cells. The quantum gene becomes inserted into the nuclear DNA of the Beta cell. Transcription machinery present in the nucleus transcribes the quantum gene. Messenger RNAs generated by transcribing the exogenous quantum genes enhances the Beta cells' production of insulin molecules. The increase in insulin production by Beta cells successfully manages diabetes mellitus.

The transcribable genetic information linked to the unique identifier may occur in the form of naturally found transcribable genetic information or may occur as artificially created transcribable genetic information, referred to as ‘artificial transcribable genetic information’. Naturally found transcribable genetic information would be a segment of transcribable genetic information that would be found in a cell's genome otherwise referred to as a gene. Artificial transcribable genetic information would be transcribable genetic information that would represent either (i) a modified form of a naturally occurring gene or (ii) a segment of nucleotides that represents transcribable genetic information that is artificially created to produce a medically beneficial result.

A quantum gene, as it exists as a functional part of the deoxyribonucleic acid of a cell, is a segment of deoxyribonucleic acid, comprised of both a unique identifier and a segment of transcribable biologic information, that is capable of being inserted into a cell's nuclear DNA. DNA is comprised of two parallel strands of nucleotides. Each strand of DNA is a mirror image of each other since adenine must combine with thymine and cytosine must combine with guanine. Therefore since each strand of DNA is a mirror image of each other, one strand of DNA possesses the nucleotide sequence that codes for both strands; one strand represents the DNA code, while the second strand represents the mirror image of the first strand. In this manner, a quantum gene can be defined in its most elemental form as a sequence of nucleotides comprising a single strand of nucleotides.

A quantum gene could thus be represented as a single strand of nucleotides comprised of the nucleotides adenine, cytosine, guanine and thymine. The double stranded form of a quantum gene would be the single strand of nucleotides attached in parallel to a second strand of nucleotides that represents the mirror image of the single strand of nucleotides. Double stranded deoxyribonucleic acid segments is the form quantum genes take when a quantum gene is inserted into a cell's nuclear genome.

### Conclusions, Ramification, and Scope

A ‘quantum gene’ is comprised of a sequence of nucleotides that represents a ‘unique identifier’ physically linked to a sequence of nucleotides that represent a discrete quantity of genetic information; these sequences of nucleotides being comprised of some combination of the nucleotides being referred to by their nitrogenous base as adenine (A), thymine (T), cytosine (C), and guanine (G). The genetic information associated with the above-mentioned unique identifier may be comprised of a portion of transcribable genetic information and a portion of nontranscribable genetic information which together define a specific gene, otherwise referred to as a discrete quantity of genetic information.

Accordingly, the reader will see that the concept and utilization of the quantum gene has never before been recognized nor appreciated by those skilled in the art.

Although the description above contains specificities, these should not be construed as limiting the scope of the invention but as merely providing illustrations of some of the presently preferred embodiments of the invention.

Thus the scope of the invention should be determined by the appended claims and their legal equivalents, rather than by the examples given.

### Number of Drawings: 0

The terms and expressions which are employed here are used as terms of description and are not of limitation and there is no intention, in the use of terms and expressions, of excluding equivalents of the features presented, and described, or portions thereof, it being recognized that various modifications are possible in the scope of the invention or process as claimed.

What is claimed:

1. Quantum gene comprised of
(a) a quantity of nucleotides which represent a unique identifier, and
(b) a quantity of nucleotides which represent genetic information.

2. The genetic information in claim 1 wherein said genetic information is comprised of a quantity of nucleotides which represent transcribable genetic information and a quantity nucleotides which represent nontranscribable genetic information,
whereby said quantity of nucleotides that represent transcribable genetic information, upon being transcribed, produce a form of ribonucleic acid, whereby said quantity of nucleotides that represent nontranscribable genetic information, which might represent a sequence of nucleotides that act as spacers or represents genetic command functions, such a STOP code, which said STOP code signals to a transcription complex to cease transcription.

3. The unique identifier in claim 1 wherein said unique identifier is a unique sequence of nucleotides which represents a unique identification associated with said genetic information.

4. The unique identifier in claim 1 wherein said unique identifier is physically connected to said genetic information along a nucleotide strand on the side of said nucleotide strand where a transcription complex assembles along said nucleotide strand and begins to transcribe said genetic information.

5. The unique identifier in claim 1 wherein said unique identifier is a segment of nucleotides comprised of a unique array of nucleotides that represent a naturally occurring unique identifier of said genetic information or represents an artificial unique identifier of said genetic information,
whereby naturally occurring genetic information already has a unique identifier that can be used by a transcription complex to locate said naturally occurring genetic information once an exogenously produced said naturally occurring genetic information is inserted into a cell's nuclear genome,
whereby artificially created genetic information would require an artificial unique identifier in order for a transcription complex to locate said artificial genetic information once said exogenously produced artificial genetic information is inserted into said cell's nuclear genome.

6. The genetic information in claim 1 wherein said genetic information is comprised of a quantity of nucleotides, which when transcribed by said cell's transcription complex produces a quantity of ribonucleic acid molecules.

7. The ribonucleic acid molecules in claim 6 wherein said ribonucleic acid molecules may be precursor ribonucleic acid molecules that require modification by nuclear enzymes prior to being translatable or may be ribonucleic acid molecules that are directly translatable without further modification.

8. The quantity of nucleotides which represent a unique identifier in claim 1 wherein said quantity of nucleotides which represent a unique identifier is physically attached to, but separated from said quantity of nucleotides which represent said genetic information by a quantity of nucleotides that do not represent said unique identifier and do not represent said genetic information,
whereby the space between said unique identifier and said genetic information may facilitate the attachment of said transcription complex to said segment of nucleotides comprising said quantum gene.

9. The genetic information in claim 1 wherein said genetic information when transcribed produces precursor ribonucleic acid molecules that require modification by enzymes in order to become functional ribonucleic acid molecules or said genetic information when transcribed produces ribonucleic acid molecules that are fully functional when said genetic information is transcribed.

10. The genetic information in claim 1 wherein said genetic information when transcribed produces a quantity of ribonucleic acid molecules found in nature and produces a quantity of ribonucleic acid molecules not found in nature, said quantity of ribonucleic acid molecules not found in nature are artificially created to perform a medically beneficial function.

11. The nucleotides in claim 1 wherein said nucleotides are comprised of a quantity of adenine nucleotides, a quantity of cytosine nucleotides, a quantity of guanine nucleotides, and a quantity of thymine nucleotides.

12. The quantum gene in claim 1 wherein said quantum gene is a segment of deoxyribonucleic acid.

13. The quantity of nontranscribable genetic information in claim 2 wherein said quantity of nontranscribable genetic information may be dispersed in different locations throughout said nucleotide sequence of said transcribable genetic information,
whereby said quantity of nucleotides represent nontranscribable genetic information which represents a quantity of said genetic commands such as said STOP codes, which may be dispersed in different locations along said nucleotide sequence of said transcribable genetic information which cellular elements may influence whether said transcription complex which is transcribing said translatable genetic information actually ceases transcription at that point where said nontranscribable genetic information is located or continues transcribing past said STOP code,
whereby this represents a means by the cell to modify the production of proteins to produce differing types of proteins to meet the cell's needs given the presence of said cellular elements which may influence said transcription complex.

14. A method to produce a quantum gene comprised of
(a) combining together a quantity of nucleotides which represent a unique identifier, and
(b) a quantity of nucleotides which represent genetic information.

15. The method to produce a quantum gene in claim 14 wherein said genetic information is comprised of a quantity of nucleotides which represent transcribable genetic information and a quantity nucleotides which represent nontranscribable genetic information,
whereby said quantity of nucleotides that represent transcribable genetic information, upon being transcribed, produce a form of ribonucleic acid, whereby said quantity of nucleotides that represent nontranscribable genetic information, which might represent a sequence of nucleotides that act as spacers or represents genetic command functions, such a STOP code, which said STOP code signals to a transcription complex to cease transcription.

16. The method to produce a quantum gene in claim 14 wherein said unique identifier is a unique sequence of nucleotides which represents a unique identification associated with said genetic information.

17. The method to produce a quantum gene in claim 14 wherein said unique identifier is physically connected to said genetic information along a nucleotide strand on the side of said nucleotide strand where a transcription complex assembles along said nucleotide strand and begins to transcribe said genetic information.

18. The method to produce a quantum gene in claim 14 wherein said unique identifier is a segment of nucleotides comprised of a unique array of nucleotides that represent a naturally occurring unique identifier of said genetic information or represents an artificial unique identifier of said genetic information,
whereby naturally occurring genetic information already has a unique identifier that can be used by a transcription complex to locate said naturally occurring genetic information once an exogenously produced said naturally occurring genetic information is inserted into a cell's nuclear genome,
whereby artificially created genetic information would require an artificial unique identifier in order for a transcription complex to locate said artificial genetic information once said exogenously produced artificial genetic information is inserted into said cell's nuclear genome.

19. The genetic information in claim 18 wherein said genetic information is comprised of a quantity of nucleotides, which when transcribed by said cell's transcription complex produces a quantity of ribonucleic acid molecules.

20. The ribonucleic acid molecules in claim 19 wherein said ribonucleic acid molecules may be precursor ribonucleic acid molecules that require modification by nuclear enzymes prior to being translatable or may be ribonucleic acid molecules that are directly translatable without further modification.
